SEQUENCE LISTING (8 sequences)

SEQ ID NO: 1 - DNA, 20 bp, Artificial Sequence
  GAPDH forward primer
ttgaggtcaatgaaggggtc

SEQ ID NO: 2 - DNA, 20 bp, Artificial Sequence
  GAPDH reverse primer
tcgtcccgtagacaaaatgg

SEQ ID NO: 3 - DNA, 19 bp, Artificial Sequence
  ER alpha forward primer
cctcccgccttctacaggt

SEQ ID NO: 4 - DNA, 19 bp, Artificial Sequence
  ER alpha reverse primer
cacacggcacagtagcgag

SEQ ID NO: 5 - DNA, 23 bp, Artificial Sequence
  ER beta forward primer
ctgtgatgaactacagtgttccc

SEQ ID NO: 6 - DNA, 21 bp, Artificial Sequence
  ER beta reverse primer
cacatttgggcttgcagtctg

SEQ ID NO: 7 - DNA, 19 bp, Artificial Sequence
  PR forward primer
ctccgggaccgaacagagt

SEQ ID NO: 8 - DNA, 21 bp, Artificial Sequence
  PR reverse primer
acaacaaccctttggtagcag

(19) United States
(12) Patent Application Publication
(10) Pub. No.: US 2020/0332360 A1
(43) Pub. Date: Oct. 22, 2020

(54) METHOD AND KIT FOR DIAGNOSING PRETERM BIRTH

(71) Applicant: KOREA UNIVERSITY RESEARCH AND BUSINESS FOUNDATION, Seoul (KR)

(72) Inventors: Ki Hoon AHN, Seoul (KR); Hee Youn KIM, Seoul (KR)

(21) Appl. No.: 16/850,412

(22) Filed: Apr. 16, 2020

(30) Foreign Application Priority Data
Apr. 16, 2019 (KR) 10-2019-0044448

(51) Int. Cl. C12Q 1/6876; A61B 17/42

(52) U.S. Cl. CPC C12Q 1/6876; A61B 2017/4225; C12Q 2600/158; A61B 17/42

(57) ABSTRACT

Disclosed are: a method for measuring the expression level of an ERa or PR gene to provide information necessary for diagnosis of preterm birth; and a kit for diagnosis of preterm birth, the kit including a probe or primers for an ERa or PR gene and an antibody to an ERa or PR protein, so that reliable information for diagnosis of preterm birth can be simply and easily provided.

### FIELD

The present disclosure was made with the support of the Ministry of Science and ICT in Korea under Project No. 2014R1A1A1002300, which was conducted in the program entitled “Leading Search” in the project named “Development of animal model for cervical deficiency and injury-related preterm birth and its validation”, by Korea University Research and Business Foundation, under management of the National Research Foundation of Korea, from 1 May 2014 to 30 Apr. 2017.

This application claims priority to and the benefit of Korean Patent Application No. 10-2019-0044448 filed in the Korean Intellectual Property Office on 16 Apr. 2019, the disclosure of which is incorporated herein by reference.

The present disclosure relates to a method and a kit for diagnosing and treating preterm birth.

### BACKGROUND

Human births in the 20th to 37th week of pregnancy is called preterm birth, and preterm birth accounts for 5-18% of all human births. About 75% of death cases before and after birth are due to preterm birth, and premature newborn babies are at high risk of neurological, respiratory, and gastrointestinal complications. Therefore, it is very important to well understand the procedures necessary for maintenance of pregnancy and the potential causes of preterm birth. Preterm birth is due to causes, such as preterm birth experience, multiple pregnancy, cervical dysfunction, smoking, abnormal body mass index, alcohol consumption, maternal age, stress, and genetic factors. The above factors that affect preterm birth have been known, but a method capable of simply and easily diagnosing preterm birth has not been developed so far.

### PRIOR ART DOCUMENT

### Non-Patent Document

Romero R et al., Preterm labor: one syndrome, many causes. Science 2014; 345: 760-5

### SUMMARY

The present inventors have conducted research and endeavored to develop a method for determining a risk of preterm birth and making an easy and simple diagnosis of preterm birth. As a result, the present inventors have established that mothers with preterm birth have a tendency of a significantly increased expression of the estrogen alpha receptor gene in the smooth muscle tissue of the proximal cervix and a significantly decreased expression of the progesterone receptor gene in the epithelial tissue of the proximal cervix and, as a result, the present inventors have completed the present disclosure.

An aspect of the present disclosure is to provide a method for providing information necessary for diagnosis of preterm birth, the method including measuring the expression level of an estrogen receptor alpha (ERα) or progesterone receptor (PR) gene in a biological sample isolated from the cervix of a subject.

Another aspect of the present disclosure is to provide a kit for diagnosis of preterm birth, the kit including: a primer or probes specifically binding to a gene sequence of an estrogen receptor alpha (ERα) or progesterone receptor (PR); or an antibody specifically binding to a protein of estrogen receptor alpha (ERα) or progesterone receptor (PR).

Still another aspect of the present disclosure is to provide a method for preventing or treating preterm birth.

In accordance with an aspect of the present disclosure, there is provided a method for providing information necessary for diagnosis of preterm birth, the method including measuring the expression level of an estrogen receptor alpha (ERα) or progesterone receptor (PR) gene in a biological sample isolated from the cervix of a subject.

As used herein, the term “subject” refers to a subject that is presumed to have a possibility of preterm birth and thus desires to obtain information on a risk of preterm birth, and the term indicates a fertile mammal. Specifically, the mammal includes, but is not limited to, dogs, cats, horses, cows, pigs, mice, rats, or humans.

In a specific embodiment of the present disclosure, the subject is an animal model of preterm birth, and more specifically, a mouse/rat model of preterm birth, which is characterized by undergoing a cervical excision and/or LPS administration. A method for fabricating an animal model of preterm birth according to a specific embodiment of the present disclosure is disclosed in Korean Patent Publication No. 10-2017-0125708.

The present inventors have found that there is a tendency in the occurrence of preterm birth that the expression of the estrogen alpha receptor gene is significantly increased in the smooth muscle tissue of the proximal cervix and the expression of the progesterone receptor gene is significantly decreased in the epithelial tissue of the proximal cervix. Therefore, the present disclosure is based on a correlation between the occurrence of preterm birth and an increased expression level of the estrogen alpha receptor gene in the smooth muscle tissue of the proximal cervix or a decreased expression level of the progesterone receptor gene in the epithelial tissue of the proximal cervix.

Therefore, in a specific embodiment of the present disclosure, the expression level of the estrogen receptor alpha gene may be measured in the smooth muscle tissue of the proximal cervix of the subject.

In another specific embodiment of the present disclosure, the expression level of the progesterone receptor gene may be measured in the epithelial tissue of the proximal cervix of the subject.

In an embodiment of the present disclosure, the present disclosure further includes a step of comparing the measured expression level of the ERa or PR gene in the subject with the expression level of the gene measured in a biological sample of a normal control.

In a specific embodiment of the present disclosure, the measuring of the expression level of the ERa or PR gene is conducted by a measurement of the mRNA or protein expression level of the gene.

In the method of the present disclosure, the measurement of the mRNA expression level of the ERa or PR gene is carried out by a step of amplifying mRNA of the ERa or PR gene via RT-PCR.

In a specific embodiment of the present disclosure, RT-PCR for measuring the mRNA expression level of ERa is performed using a primer pair consisting of the nucleotide sequences of SEQ ID NO: 3 and SEQ ID NO: 4.

In another specific embodiment of the present disclosure, RT-PCR for measuring the mRNA expression level of PR is performed using a primer pair consisting of the nucleotide sequences of SEQ ID NO: 7 and SEQ ID NO: 8.

In the method of the present disclosure, the measurement of the protein expression level of the ERa or PR gene is carried out by a step of performing an immunoassay of a protein of the ERa or PR gene.

In an embodiment of the present disclosure, the method of the present disclosure further includes a step of determining that the subject has a high risk of preterm birth when the measured expression level of the ERa gene in the subject is higher than that in the normal control.

In another embodiment of the present disclosure, the method of the present disclosure further includes a step of determining that the subject has a high risk of preterm birth when the measured expression level of the PR gene of the subject is lower than that in the normal control.

In still another embodiment of the present disclosure, the method of the present disclosure further includes measuring the level of estrone (E1) or progesterone (P4) in a biological sample isolated from the blood of the subject.

Furthermore, the method of the present disclosure further includes comparing the level of E1 or P4 measured in the step with the level of E1 or P4 measured in a biological sample isolated from the blood of the normal control.

In a more specific embodiment of the present disclosure, it is determined that the subject has a higher risk of preterm birth when the levels of estrone and progesterone in the subject are lower than those in the normal control.

In the method of the present disclosure, the determination of a risk of preterm birth through the measurement of the expression level of the ERa or PR gene may be largely carried out in two manners, that is, genetic analysis and immunoassay.

When the present disclosure is performed on the basis of genetic analysis, a probe or primers specifically binding to the nucleotide sequence of the ERa or PR gene are used. In a case of using primers, a genetic amplification reaction is performed to investigate the expression level of the ERa or PR gene. Since the expression level of the ERa or PR gene is analyzed in the present disclosure, a sample (e.g., cells or tissue) from a subject of analysis is investigated for the ERa or PR mRNA level, thereby determining the expression level of the ERa or PR gene. Therefore, in the present disclosure, a gene amplification reaction is basically performed using: mRNA in a sample as a template; and primers binding to mRNA or cDNA. In order to obtain mRNA, total RNA is first isolated from the sample. The isolation of total RNA may be performed by a conventional method known in the art. For example, total RNA in cells may be easily isolated using Trizol. Then, cDNA is synthesized from the isolated mRNA, and the cDNA is amplified. Since the total RNA of the present disclosure is isolated from a sample of humans, a poly-A tail is located at an end of mRNA. Therefore, an oligo dT primer using this sequence characteristics and a reverse transcriptase can be used to easily synthesize cDNA. Then, the synthesized cDNA is amplified through a gene amplification reaction.

The primer used in the present disclosure is hybridized or annealed with one region of the template to form a double-chain structure. The nucleic acid hybridization conditions suitable for forming such a double-chain structure are disclosed in known literature, such as Joseph Sambrook, et al., Molecular Cloning, A Laboratory Manual, Cold Spring Harbor Laboratory Press, Cold Spring Harbor, N.Y. (2001).

Various DNA polymerases may be used in the amplification of the present disclosure and include the “Klenow” fragment of E. coli DNA polymerase I, thermostable DNA polymerase, and bacteriophage T7 DNA polymerase. Preferably, the polymerase is thermostable DNA polymerase that can be obtained from a variety of bacteria species, which include Thermus aquaticus (Taq), Thermus thermophilus (Tth), Thermus filiformis, Therm is flavus, Thermococcus literalis, and Pyrococcus furiosus (Pfu).

When a polymerization reaction is conducted, the components necessary for such a reaction are preferably provided in excess in a reaction vessel. The excess with respect to the components necessary for the amplification reaction means an amount of each component such that the amplification reaction is not substantially restricted by the concentration of the component. A cofactor, such as Mg²⁺, and dATP, dCTP, dGTP, and dTTP are required to be provided to the reaction mixture to such an extent that a desired degree of amplification can be achieved.

All enzymes used in the amplification reaction may be in an active state under the same reaction conditions. Actually, a buffer allows all the enzymes to approach to optimal reaction conditions. Therefore, the amplification procedure of the present disclosure may be carried out in a single reactant without any change in reaction conditions, such as reactant addition.

The term “amplification reaction” described herein refers to a reaction that amplifies nucleic acid molecules. Various amplification reactions have been reported in the art, and include, but are not limited to, polymerase chain reaction (hereinafter, PCR), reverse transcription-polymerase chain reaction (hereinafter, RT-PCR), ligase chain reaction (LCR), Gap-LCR, repair chain reaction, transcription-mediated amplification (TMA), self-sustained sequence replication, selective amplification of target polynucleotide sequences, consensus sequence primed polymerase chain reaction (CP-PCR), arbitrarily primed polymerase chain reaction (AP-PCR), nucleic acid sequence based amplification (NASBA), strand displacement amplification, and loop-mediated isothermal amplification (LAMP).

According to a specific embodiment of the present disclosure, the primers used in the amplification reaction are oligonucleotides having sequences complementary to the cDNA sequence of the ERa or PR gene. The sequences of the primer need not be perfectly complementary to a partial sequence of the template, and the sequences of the primers are sufficient as long as the sequences of the primers are sufficiently complementary within the range in which the primers can be hybridized with the template to make inherent actions thereof. Therefore, the primer pair of the present disclosure need not have a perfectly complementary sequence to the ERa or PR cDNA sequence, and the primer pair of the present disclosure is sufficient as long as the primer pair is sufficiently complementary within the range in which the primer pair can be hybridized with the sequence and act as primers.

The primers of the present disclosure may be prepared to have complementary sequences to the mRNA (i.e., cDNA) sequence of the ERa or PR gene. These primers can be easily designed by a person skilled in the art with reference to the cDNA sequence of the ERa or PR gene. The nucleic acid molecule thus amplified is analyzed by an appropriate method, thereby investigating the expression level of the ERa or PR gene. For example, the above-described amplification reaction product is subjected to gel electrophoresis, and the resultant bands are observed and analyzed to investigate the expression level of the ERa or PR gene. When the expression level of the ERa gene is confirmed to be higher than that in a sample isolated from normal human cervix through the amplification reaction, the risk of preterm birth is determined to be high.

When the expression level of the PR gene is confirmed to be lower than that in a sample isolated from the cervix of a normal person through the amplification reaction, the risk of preterm birth is determined to be high.

Alternatively, the method of the present disclosure can be performed through hybridization-based assay using a probe hybridized with the ERa or PR gene or cDNA of the ERa or PR gene. As used herein, the term “probe” is a single-chain nucleic acid molecule, and contains a nucleotide sequence complementary to a target nucleic acid sequence. According to an embodiment of the present disclosure, the probe of the present disclosure can be modified within a range in which an advantage of the probe of the present disclosure, that is, an improvement in hybridization specificity is not impaired. These modifications, that is, labels, can provide signals enabling the detection of hybridization or not, and can be linked to an oligonucleotide. Appropriate labels include, but are not limited to, fluorophores (e.g., fluorescein, phycoerythrin, rhodamine, lissamine, and Cy3 and Cy5), chromophores, chemiluminophores, magnetic particles, radioisotopes (P³² and S³⁵), mass labels, electron-dense particles, enzymes (alkaline phosphatase or horseradish peroxidase), cofactors, substrates of enzymes, heavy metals (e.g., gold), antibodies, streptavidin, biotin, digoxigenin, and haptens having specific binding partners, such as chelating groups. The labels provide signals that are detectable by fluorescence, radioactivity, measurement of color development, mass measurement, X-ray diffraction or absorption, magnetism, enzymatic activity, mass analysis, binding affinity, high frequency of hybridization, or nanocrystals.

According to an embodiment, the probe of the present disclosure, hybridizing with cDNA of the ERa or PR gene, is immobilized on an insoluble carrier (e.g., nitrocellulose or nylon filter, glass plate, silicone, and fluorocarbon supports) to be fabricated into a microarray. The immobilization onto the insoluble carrier is carried out by a chemical bonding method or a covalent bonding method, such as UV. For example, the hybridizable array elements may be bound to a glass surface that is modified to contain an epoxy compound or an aldehyde group, or may be bound to a polylysine-coated surface by using UV. In addition, the hybridizable array element may be bound to a carrier via a linker (e.g., an ethylene glycol oligomer and a diamine).

When the probe is used, the probe is hybridized with a cDNA molecule. Appropriate hybridization conditions may be determined through a series of procedures by an optimization procedure. For example, the conditions of temperature, concentrations of components, hybridization and washing times, buffer components, and pH and ion intensity thereof, depend on various factors including the length of the probe, the GC amount, the sequence of the target nucleotide, and the like.

After the hybridization reaction, a hybridization signal generated through the hybridization reaction is detected. Hybridization signaling may be carried out by various methods depending on, for example, the type of label attached to the probe. For example, when the probe is labeled with an enzyme, a substrate of the enzyme may be reacted with a hybridization reaction product to investigate the presence or absence of hybridization. Examples of a combination of enzyme/substrate that may be used include, but are not limited to, peroxidase (e.g., horseradish peroxidase) and chloronaphthol, aminoethyl carbazole, diaminobenzidine, D-ludferin, lucigenin (bis-N-methylacridinium nitrate), resorufin benzyl ether, luminol, Amplex Red reagent (10-acetyl-3,7-dihydroxyphenoxazine), p-phenylenediamine-HCl and pyrocatechol (HYP), tetramethylbenzidine (TMB), 2,2′-azine-di[3-ethylbenzthiazoline sulfonate] (ABTS), o-phenylenediamine (OPD), and naphthol/pyronin; alkaline phosphatase and bromochloroindolylphosphate (BCIP), nitro blue tetrazolium (NBT), naphthol-AS-B1-phosphate, and ECF substrate; and glucose oxidase and nitroblue tetrazolium (t-NBT). In cases where a probe is labeled with a gold particle, the probe may be detected by a silver staining method using silver nitrate.

Alternatively, the expression level of ERa or PR protein can be measured by an immunoassay method. This immunoassay can be performed according to various immunoassay or immunostaining protocols that were developed in the present disclosure.

Examples of the immunoassay or immunostaining format include, but are not limited to, radioimmunoassay, radioimmunoprecipitation assay, immunoprecipitation assay, enzyme-linked immunosorbent assay (ELISA), capture-ELISA, inhibition or competition assay, sandwich assay, flow cytometry assay, immunofluorescence, immunohistochemistry, and immunoaffinity purification assay.

For example, when the method of the present disclosure is performed according to radioimmunoassay, an antibody labeled with a radioactive isotope (e.g., C¹⁴, I¹²⁵, P³², and S³⁵) can be used to detect ERα or PR protein. The antibodies to ERα or PR protein are polyclonal or monoclonal antibodies, and preferably monoclonal antibodies. The antibodies to ERα or PR protein can be produced by the methods that are usually conducted in the art, for example, a fusion method (Kohler and Milstein, European Journal of Immunology, 6:511-519 (1976)), a recombinant DNA method or a phage antibody library method (Clackson et al, Nature, 352:624-628 (1991) and Marks et al, J. Mol. Biol., 222:58, 1-597 (1991)). General procedures for antibody production are disclosed in detail in Harlow, E. and Lane, D., Using Antibodies: A Laboratory Manual, Cold Spring Harbor Press, New York, 1999; Zola, H., Monoclonal Antibodies: A Manual of Techniques, CRCPress, Inc., Boca Raton, Fla., 1984; and Coligan, CURRENT PROTOCOLS IN IMMUNOLOGY, Wiley/Greene, NY, 1991, the contents of which are incorporated herein by reference.

When the method of the present disclosure is performed according to ELISA, a particular embodiment of the present disclosure includes the steps of: (i) coating a sample lysate to be analyzed on the surface of a solid substrate; (ii) incubating the sample lysate together with an antibody to the ERα or PR protein, as primary antibody; (iii) incubating the product in step (ii) together with a secondary antibody conjugated to an enzyme; and (iv) determining the activity of the enzyme. Suitable examples of the solid substrate include hydrocarbon polymers (e.g., polystyrene and polypropylene), glass, metals, and gels. Examples of the enzyme conjugated to the secondary antibody includes, but are not limited to, enzymes that catalyze a color reaction, a fluorescence reaction, a luminescence reaction, or an infrared reaction, and examples thereof include alkaline phosphatase, β-galactosidase, horseradish peroxidase, luciferase, and cytochrome P450. When alkaline phosphatase is used as an enzyme conjugated to the secondary antibody, a substrate for a color reaction, such as bromo-chloro-indolyl-phosphate (BCIP), nitro blue tetrazolium (NBT), naphthol-ASB1-phosphate, or enhanced chemifluorescence (ECF), may be used. When horseradish peroxidase is used as an enzyme conjugated to the secondary antibody, a substrate, such as chloronaphthol, aminoethyl carbazole, diaminobenzidine, D-luciferin, lucigenin(bis-N-methylacridinium nitrate), resorufin benzyl ether, luminol, Amplex Red reagent (10-acetyl-3,7-dihydroxyphenoxazine), HYR, TMB, ABTS, o-phenylenediamine (OPD), and naphthol/pyronin, glucose oxidase and t-NBT, and m-PMS, may be used.

When the method of the present disclosure is performed by capture-ELISA, a particular embodiment of the present disclosure includes the steps of: (i) coating an antibody to ERα or PR protein as a capturing antibody on a surface of a solid substrate; (ii) incubating the capturing antibody and a sample lysate; (iii) incubating the product in step (ii) together with a detecting antibody, which is conjugated to a label generating a signal and specifically reacts with ERα or PR protein; and (iv) measuring the signal generated from the label.

The detecting antibody has a label that generates a detectable signal. Examples of the label include, but are not limited to, chemicals (e.g., biotin), enzymes (alkaline phosphatase, β-galactosidase, horse radish peroxidase, and cytochrome P450), radioactive substances (e.g., C¹⁴, I¹²⁵, P³², and S³⁵), fluorescent substances (e.g., fluorescein), light-emitting substances, chemiluminescent substances, and fluorescence resonance energy transfer (FRET). The other various labels and labeling methods are known in the art.

In ELISA and capture-ELISA, the final measurement of the enzyme activity or signal may be carried out according to various methods known in the art.

According to another embodiment of the present disclosure, the present disclosure provides a kit for diagnosis of preterm birth, the kit including: a primer or probes specifically binding to a gene sequence of estrogen receptor alpha (ERα) or progesterone receptor (PR); or an antibody specifically binding to a protein of estrogen receptor alpha (ERα) or progesterone receptor (PR).

When the kit for diagnosis of preterm birth of the present disclosure is applied to a PCR amplification procedure, the kit of the present disclosure may optionally contain reagents necessary for PCT amplification, for example, DNA polymerase (e.g., thermally stable DNA polymerase obtained from Thermus aquaticus (Taq), Thermus thermophilus (Tth), Thermus filiformis, Therm is flavus, Thermococcus literalis, or Pyrococcus furiosus (Pfu)), DNA polymerase cofactor, and dNTPs.

In a specific embodiment of the present disclosure, a primer pair consisting of the nucleotide sequences of SEQ ID NO: 3 and SEQ ID NO: 4 may be used as primers specifically binding to the gene sequence of the estrogen acceptor alpha of the present disclosure; and a primer pair consisting of the nucleotide sequences of SEQ ID NO: 7 and SEQ ID NO: 8 may be used as primers specifically binding to the gene sequence of the progesterone receptor of the present disclosure.

When the kit for diagnosis of preterm birth is applied to the immunoassay in the present disclosure, the kit of the present disclosure may optionally include a secondary antibody and a label substrate.

The kit of the present disclosure may be fabricated by a plurality of separate packaging or compartments including the foregoing reagent components.

In accordance with still another aspect of the present disclosure, there is provided a method for prevention or treatment of preterm birth, the method including:

(a) measuring the expression level of an estrogen receptor alpha (ERα) or progesterone receptor (PR) gene in a biological sample isolated from the cervix of a subject;

(b) determining that the subject has a high risk of preterm birth when the expression level of the PR gene in the subject is lower than that in a normal control, wherein the expression level of the PR gene is measured in a biological sample from the epithelium of the cervix of the subject; and

(c) performing a prevention or treatment for preterm birth on the subject determined to have a high risk of preterm birth in step (b).

As used herein, the term “prevention” refers to all acts that inhibit or delay the occurrence of preterm birth, and the term “treatment” refers to all acts that alleviate or beneficially modify preterm birth.

As used herein, the term “administration” or “administer” refers to a direct administration of a therapeutically effective amount of a medicine necessary for prevention or treatment of preterm birth of the present disclosure into a subject (an individual), thereby forming the same amount thereof in the body of the subject.

The term “therapeutically effective amount” refers to a content of the composition, which is sufficient to provide a therapeutic or preventive effect to a subject, to which the medicine is to be administered, and thus the term has a meaning including “prophylactically effective amount.”

In an embodiment of the present disclosure, the prevention or treatment of preterm birth is carried out by administration of an agent for prevention or treatment of preterm birth or by surgical intervention.

The prevention or treatment agent for preterm birth is a tocolytic agent, an antibiotic agent, a steroid preparation, a progesterone preparation, a progesterone receptor agonist, an estrogen receptor alpha inhibitor, or a combination of thereof.

The tocolytic agent is a beta-sympathomimetic agent, a calcium channel blocker, an oxytocin antagonist, a non-steroidal anti-inflammatory drug, magnesium sulfate, or a combination thereof.

In an embodiment of the present disclosure, examples of the beta-sympathomimetic agent include, but are not limited to, ritodrine, terbutaline, fenoterol, salbutamol, albuterol, or hexoprenaline.

In an embodiment of the present disclosure, examples of the calcium channel blocker include, but are not limited to, nifedipine or amlodipine.

In an embodiment of the present disclosure, examples of the oxytocin antagonist include, but are not limited to, atosiban.

In an embodiment of the present disclosure, examples of the non-steroidal anti-inflammatory drug include, but are not limited to, indomethacin or sulindac.

The antibiotic agent is known to be administered to prevent infectious diseases in newborn babies when preterm birth is imminent, and examples thereof include all various types of antibiotic agents that can be safely used for the same purpose in the art.

The steroid preparation is administered to promote fetal lung maturation in less than 34 weeks of pregnancy. The use of steroids is known to improve the prognosis of premature infants since steroids can reduce respiratory distress syndrome, necrotizing enterocolitis, intraventricular bleeding, and overall perinatal death.

The administration of the present disclosure may be carried out via various routes, and for example, the administration may be carried out by oral, intraperitoneal, rectal or intravenous, arterial, muscle, inhalation, transdermal, subcutaneous, intradermal, intrauterine, vaginal, dural, or intracerebroventricular injection.

The administration of the prevention or treatment agent for preterm birth of the present disclosure can be applied to not only humans, but also all mammal animals that may develop diseases involved in preterm birth, such as cows, horses, sheep, pigs, goats, camels, antelopes, dogs, cats, rabbits, and rodents (mouse, rat, hamster, and gerbil).

The progesterone receptor agonist is selected from the group consisting of ulipristal, asoprisnil, telapristone, levonorgestrel, megestrol acetate, mometasone furoate, progesterone, progestin, and a combination thereof, but is not limited thereto.

In the present disclosure, the progesterone receptor agonist is characterized by upregulating actions of progesterone receptors on the epithelium of the cervix.

In an embodiment of the present disclosure, the estrogen receptor alpha inhibitor is an estrogen receptor alpha antagonist.

In the present disclosure, the estrogen receptor alpha antagonist is selected from the group consisting of fulvestrant, ICI-164384, ethamoxytriphetol, mifepristone, bazedoxifene, broparestrol, cyclofenil, ormeloxifene, ospemifene, methyl piperidino pyrazole (MPP), clomifene, tamoxifen, raloxifene, toremifene, ICI-182780, trans-hydroxytamoxifen, lasofoxifene, and a combination thereof, but is not limited thereto.

In an embodiment of the present disclosure, the surgical intervention is cervical cerclage, but is not limited thereto.

Since the method for prevention or treatment of preterm birth according to an aspect of the present disclosure includes the steps in common with the foregoing method for providing information necessary for diagnosis of preterm birth, the description of overlapping steps and constitutions applies in the same manner.

Features and advantages of the present disclosure are summarized as follows.

The present disclosure is directed to: a method for measuring the expression level of the ERα or PR gene to provide information necessary for diagnosis of preterm birth; and a kit for diagnosis of preterm birth, the kit including a probe or primers for the ERα or PR gene and an antibody to the ERα or PR protein.

The present disclosure is based on the fact that the increase in expression level of the ERα gene and the decrease in expression level of the PR gene in the proximal cervix are correlated with the occurrence of preterm birth.

According to the method of the present disclosure, reliable information for diagnosis of preterm birth can be simply and easily provided.

### BRIEF DESCRIPTION OF THE DRAWINGS

FIGS. 1A, 1B, and 1C show mRNA expressions of ERα, ERβ and PR in the cervical tissue from postpartum mice via qRT-PCR. mRNA expression of (1A) ERα, (1B) ERβ, and (1C) PR are shown. The values were expressed as the mean±SEM. *=p<0.05. ER: estrogen receptor, PR: progesterone receptor, Ex: cervical excision.

FIGS. 2A, 2B, 2C, 2D and 2E show protein expressions of ERα, ERβ, and PR in the cervical tissue, measured via western blotting. Protein expression of (2A) ERα, (2B) ERβ, (2C) PR-A, (2D) PR-B, and (2E) PR-A/PR-B are shown. The values were expressed as the mean±SEM. *=p<0.05. ER: estrogen receptor, PR: progesterone receptor, Ex: cervical excision.

FIGS. 3A, 3B, 3C, 3D, 3E, 3F, 3G, 3H and 3I show localization and expression levels of ERα, ERβ, and PR in the proximal and distal cervix determined using immunohistochemistry. (3A) ERα expression in the cervix (×200), (3B) IHC scoring of ERα in the epithelium of the cervix, (3C) IHC scoring of ERα in the smooth muscle layer of the cervix, (3D) ERβ expression in the cervix (×200), (3E) IHC scoring of ER8 in the epithelium of the cervix, (3F) IHC scoring of ERβ in the smooth muscle layer of the cervix, (3G) PR expression in the cervix (×200), (3H) IHC scoring of PR in the epithelium of the cervix, and (3I) IHC scoring of PR in the smooth muscle layer of the cervix are shown. The values were expressed as the mean±SEM. EPI: epithelium, SML: smooth muscle layer, P: proximal cervix, D: distal cervix, ER: estrogen receptor, Ex: cervical excision.

FIGS. 4A, 4B, 4C and 4D show the ER/PR expression in the proximal and distal cervix. (4A) ERα/PR expression in the cervical epithelium, (4B) ERα/PR expression in the cervical smooth muscle layer, (4C) ERβ/PR expression in the cervical epithelium, and (4D) ERβ/PR expression in the cervical smooth muscle layer are shown. EPI: epithelium, SML: smooth muscle layer, P: proximal cervix, D: distal cervix, PR: progesterone receptor, Ex: cervical excision. The values were expressed as the mean±SEM. *=p<0.05.

FIGS. 5A, 5B and 5C show a comparison of serum estrogen and progesterone levels. (5A) serum estrone (E1) level, (5B) serum 17β estradiol (E2) level, and (5C) serum progesterone (P4) level are shown. The values were expressed as the mean±SEM. *=p<0.05.

FIG. 6 shows a theoretical model for the role of cervical ERα and PR systems in cervical excision-associated preterm birth and parturition. “Muscle fatigue” may result from an increase in the expression ratio of ERα and PR, functional progesterone withdrawal, and contraction of muscle cells in the proximal cervix. Following “muscle fatigue”, the internal os (internal orifice) of the cervix is weakened and preterm birth occurs.

### DETAILED DESCRIPTION

Hereinafter, the present disclosure will be described in more detail with reference to examples. These examples are only for illustrating the present disclosure more specifically, and it would be apparent to a person skilled in the art that the scope of the present disclosure is not limited to these examples.

### Examples

Throughout the present specification, the term “%” used to express the concentration of a specific material, unless otherwise particularly stated, refers to (wt/wt) % for solid/solid, (wt/vol) % for solid/liquid, and (vol/vol) % for liquid/liquid.

Materials and Methods

1. Animal Models and Treatment

Animal experiments were approved by the Institutional Animal Care and Use Committee, Korea University (KOREA-2016-0070). Sexually mature C57BU6 mice were used. The mice in the sham group were injected with normal saline (NS) on day 16 of gestation (n=10); the mice in the excision (Ex) group were injected with NS (n=9); the mice in the LPS group were injected with LPS (n=10); and the mice in Ex+LPS group underwent cervical excisions and LPS injections (n=10). The cervix was excised from the 5-week-old female mice. The mice underwent a cervical excision through the vagina under anesthesia with isoflurane inhalation. After 3 weeks, female mice were allowed to mate with male mice. The day on which a mucous plug was observed after mating was set as “Day 1 of gestation”. The animals were given water and food ad libitum, and they were housed under controlled 12-h light/12-h dark conditions at 22-24° C. On day 16 of gestation, the mice were administered with NS (100 μl) or LPS dissolved in NS (100 μg/100 μl). The mice were then anaesthetized with isoflurane, and LPS was injected into the cavity between the first and second gestational sacs after an abdominal incision. The subcutaneous tissues were closed with Vicryl, and the skin was closed with black silk.

2. Tissue Collection

Immediately after parturition, the cervix and blood were collected. The cervix was fixed in 4% buffered formalin at room temperature for 24 hours and embedded in paraffin for immunohistochemical analysis. Parts of the cervix were placed in liquid nitrogen and stored −80° C. before use for RNA and protein extraction.

3. RNA Extraction, cDNA Synthesis, and Quantitative Real-Time PCR

Total RNA was extracted after the cervix of frozen mice was homogenized in TRIzol solution (79306; Qiagen, CA, USA). RNA concentration was measured, and the purity thereof was checked at 260/280 nm using NanoDrop spectrophotometer (Thermo scientific, Massachusetts, USA). cDNA synthesis was performed using 1 μg of total RNA. PCR amplification was performed using SUBR Green (TOPreal qPCR 2□PreMIX; Enzynomics, Daejeon, South Korea). Quantitative real-time PCR was performed through CFX96™ real-time system (Bio-Rad, CA, USA). qRT-PCR analysis was repeatedly performed three times per sample. Sequences of primers for target genes are shown in Table 1 below.

TABLE 1
Mouse primer sequences used for quantitative
real-time PCR

| | | | SEQ |
|---|---|---|---|
| | | | ID |
| Gene | Direction | Sequence (5′ to 3′) | NO |
| GAPDH | Forward | TTGAGGTCAATGAAGGGGTC | 1 |
| | Reverse | TCGTCCCGTAGACAAAATGG | 2 |
| ERα | Forward | CCTCCCGCCTTCTACAGGT | 3 |
| | Reverse | CACACGGCACAGTAGCGAG | 4 |
| ERβ | Forward | CTGTGATGAACTACAGTGTTCCC | 5 |
| | Reverse | CACATTTGGGCTTGCAGTCTG | 6 |
| PR | Forward | CTCCGGGACCGAACAGAGT | 7 |
| | Reverse | ACAACAACCCTTTGGTAGCAG | 8 |

4. Protein Extraction and Western Blot

The cervix of pregnant mice injected with NS or LPS was homogenized in RIPA lysis buffer containing a protease inhibitor cocktail (150 mM NaCl, 1% Triton X-100, 1% SD, 0.1% SDS, 50 mM Tris-HCl, 2 mM EDTA; Biosesang, Sungnam, South Korea). The tissue was centrifuged at 19,000 g for 15 minutes at 15° C. The protein concentration was quantified using Bradford assay (Protein assay dye reagent concentrate buffer; Bio-Rad, CA, USA). Proteins (20 μg per lane) were separated on 8% SDS PAGE gel under electrophoresis at 100 V. The pre-stained marker (161-0374; Bio-Rad, CA, USA) was included for size distinguishment. The electrophoresis-completed gel was transferred on the nitro cellulose membrane at 100 V for 120 minutes. The membrane was blocked with 3% skim milk, diluted with TBS-T (Tris-buffered saline solution and Tween 20), treated with primary antibody, and then incubated overnight at 4° C.: ERα (1:1000, sc8005; Santa Cruz, Tex., USA), ERβ (1:1000, sc8974; Santa Cruz), PR (1:250, MA1-410; Invitrogen, CA, USA), HPRT (1:1000, sc376559; Santa Cruz), and β-actin (1:1000, sc47778; Santa Cruz). The membrane incubated with the primary antibody was washed with TBS-T (pH 7.4), and incubated with HRP-conjugated anti-mouse and anti-rabbit secondary antibodies (1:5000; Jackson Immuno Research, PA, USA). The bands were detected using chemiluminescence. The blots were developed using ECL kit (SuperSignal West Pico PLUS Chemiluminescent Substrate; Thermo scientific, Massachusetts, USA), exposed to a medical X-ray film (EA8EC; Agra-Gevaert, Mortsel, Belgium), and then analyzed using Image J software.

5. Immunohistochemistry and Scoring

Histology of the tissue fixed in 4% buffered formalin for 1 hour and embedded in paraffin block was analyzed as follows. The paraffin block was cut into 4-μm sections, mounted on slides coated with silane (5116-20F; MUTO, Tokyo, Japan), and then stained. All the slides were analyzed using a microscope (BX61; Olympus, Tokyo, Japan). The sections embedded with paraffin were stained with ERα (1:35, M7047; Dako, CA, USA), ERβ (1:30, sc8974; Santa Cruz), and PR (1:70, sc538; Santa Cruz) antibodies. The sections were dewaxed with xylene, and rehydrated with concentration graded alcohol. Then, the sections were washed with tap water, heated in citrate buffer for 15 minutes, and then left at room temperature for 30 minutes. The sections were treated with 0.1% H₂O₂ for 10 minutes to block inherent peroxidase, washed with buffered saline solution (Tween 20, 1×, pH 7.4; Scytek, UT, USA), and then blocked with Super block (Scytek, UT, USA) for 10 minutes.

The levels of ERα, ERβ, and PR were measured by incubation of the fragments with primary antibodies to ERα, ERβ, and PR. Then, the fragments were washed with Tris buffered saline solution, and incubated with biotinylated secondary antibody. The color development was made by incubating the slides with DAB reagent. The fragments were counterstained with hematoxylin, dehydrated, and mounted. All the steps were carried out at room temperature. The fragments were analyzed using Rotary Microtome (RM2255; Leica, Wetzlar, Germany). Immunostaining scores were evaluated from 0 to 3 according to the level of color development. Scoring was made on the basis of the intensity of staining as follows:

Negative (0)—not stained or less than 10% stained;

Weak staining (+1)—more than 10% stained;

Weak to medium staining (+2)—more than 34% stained; and

Strong staining (+3)—more than 67% stained

6. Serum Hormone Quantification

The blood was collected, and centrifuged at 3,000 g for 10 minutes. The serum was separated, and stored at −80° C. The levels of serum estrone (E1), 17β estradiol (E2), estriol (E3), and progesterone (P4) were investigated using EILSA. The Mouse E1 ELISA kit (1:50, MBS766232; Mybiosource, CA, USA), E2 ELISA kit (ab108667; Abcam, MA, USA), E3 ELISA kit (KA0316; Abnova, Taipei, Taiwan), and P4 ELISA kit (1:300, ADI-900-011; Enzo, NY, USA) were used according to the instructions of manufacturers. The results of serum E3 are not shown, but serum E3 was not detected regardless of repeated tests.

7. Statistical Analysis

The data were expressed as mean±standard error of mean. The results of each experiment were analyzed by one-way analysis of variance (ANOVA), and the mean was compared using Tukey's and Duncan post hoc multiple comparisons. Statistical significance was set to p<0.05. The statistical analysis was performed using the SPSS software (version 13.0; SPSS Inc, Chicago, Ill.).

Results

1. mRNA Expression of ER and PR in Cervix

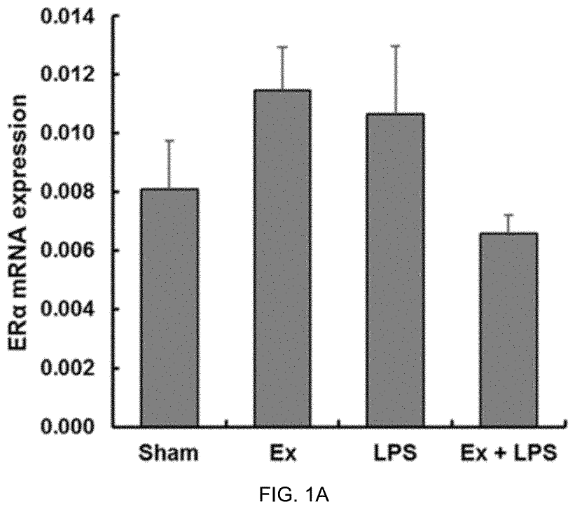

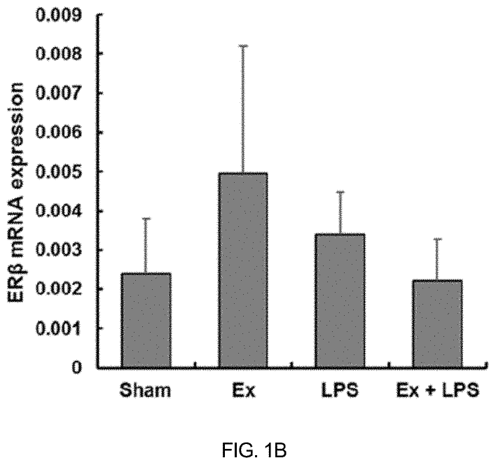

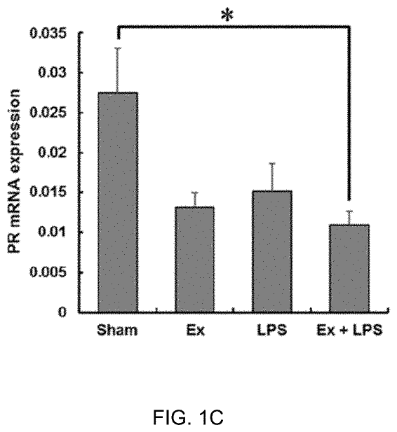

The quantitative PCR was performed to quantify the ER and PR mRNA expression in the cervix. A similar pattern was observed for both ERα and ERβ expression. ERα and ERβ expressions in the Ex and LPS groups were similar to those in the sham and the other groups (FIGS. 1A and 1B). PR expression in the cervix tended to be lower in Ex and LPS groups than in the sham group. PR expression in the cervix was significantly lower in the Ex+LPS group than in the sham group (FIG. 1C).

2. Protein Levels of ER and PR

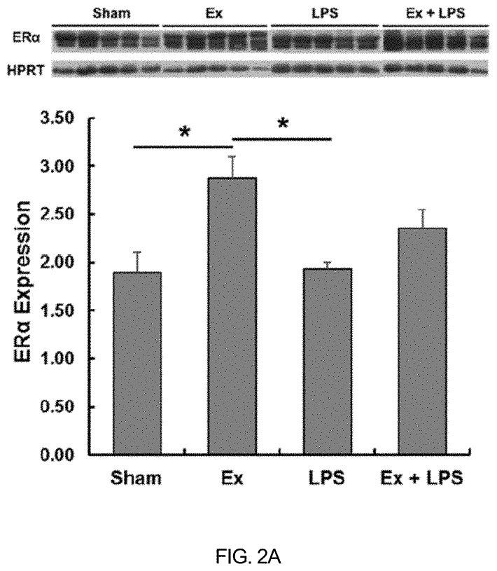

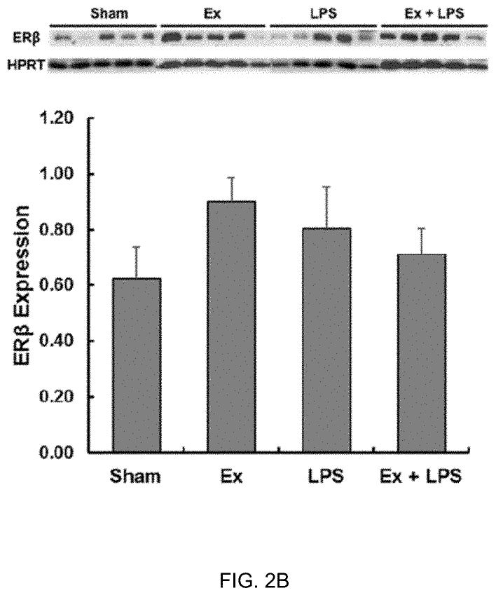

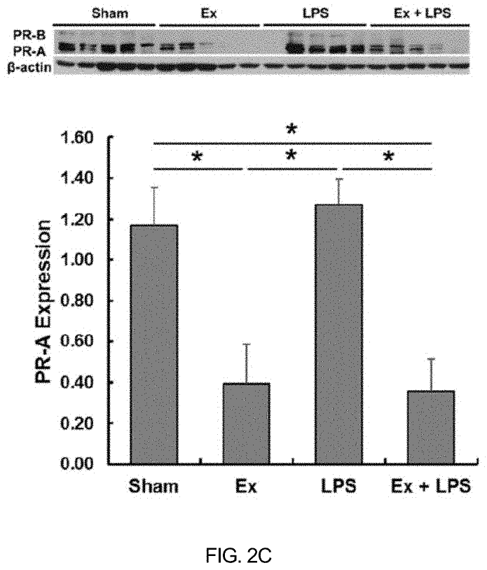

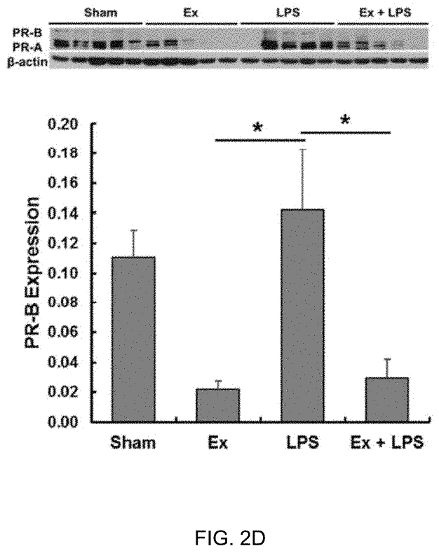

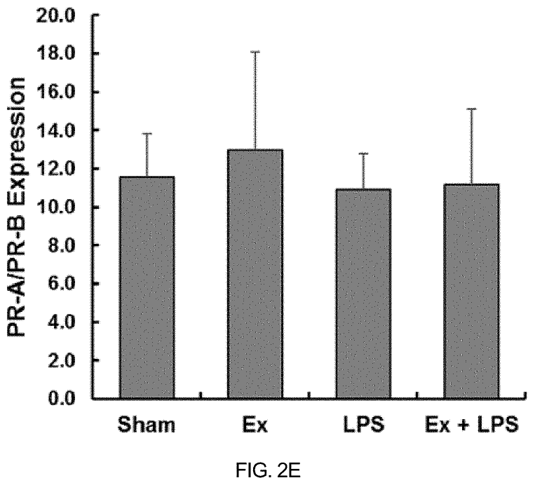

ERα expression was significantly higher in the Ex group than in the sham group and the LPS group (FIG. 2A). ERβ expression did not differ among groups (FIG. 2B). PR-A expression was significantly lower in the Ex and Ex+LPS groups than in the sham and LPS groups (FIG. 2C). PR-B expression was significantly lower in the Ex and Ex+LPS groups than in LPS group (FIG. 2D). PR-A and PR-B showed similar expression aspects among groups, and thus PR-A/PR-B among groups did not show a significant difference (FIG. 2E).

3. Localization and Expression of ER and PR Receptors in Cervix

To understand the expression trend of sexual hormone receptors in the cervix more specifically, the present inventors used immunohistochemistry. Specifically, to investigate expression levels and sites of ER and PR, the cervix was divided into proximal and distal sections to fabricate tissue sections, and three sites for section were randomly selected to evaluate ER and PR expression. The mean values of the expression levels were calculated. More specifically, the expression levels in the epithelium (EPI) and smooth muscle layer (SML) were differentially scored.

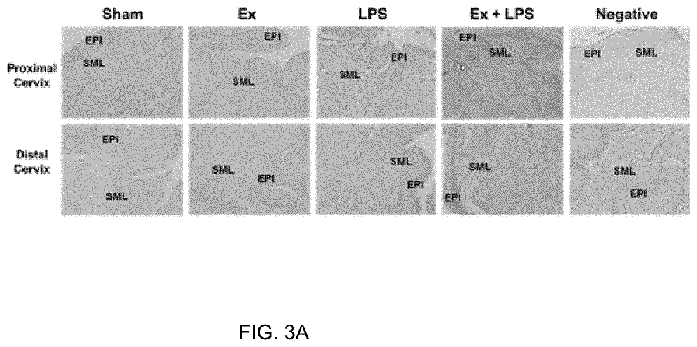

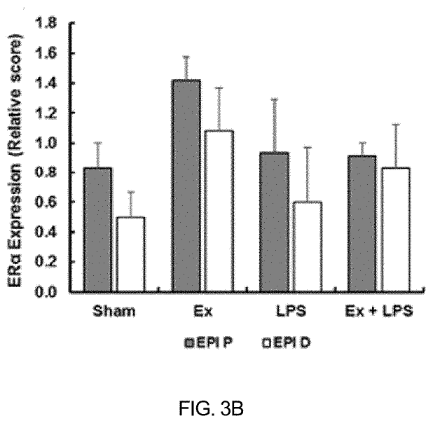

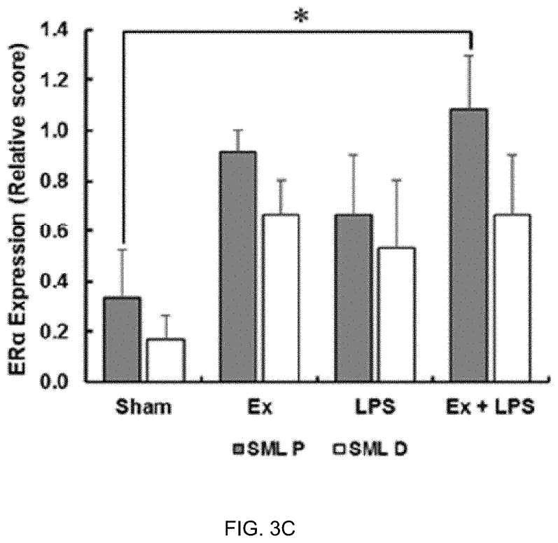

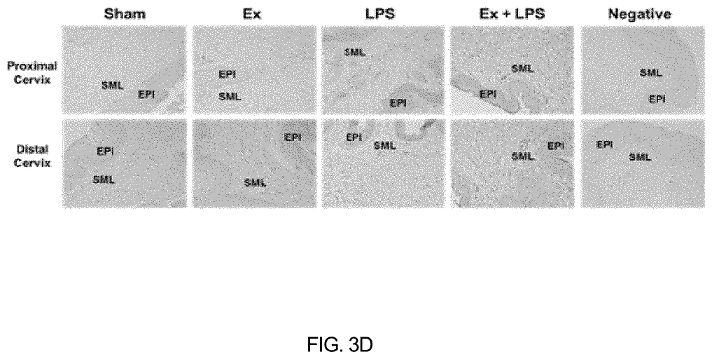

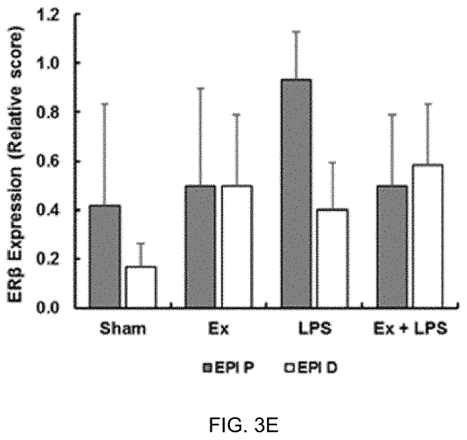

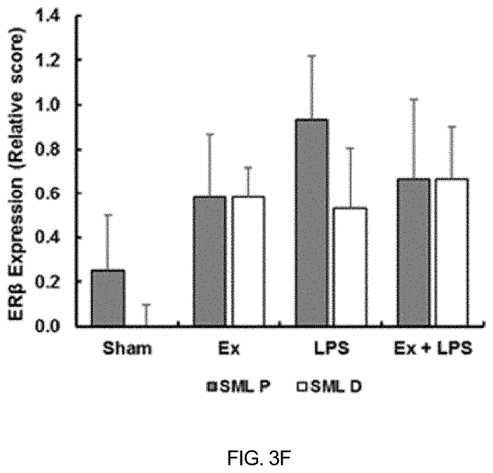

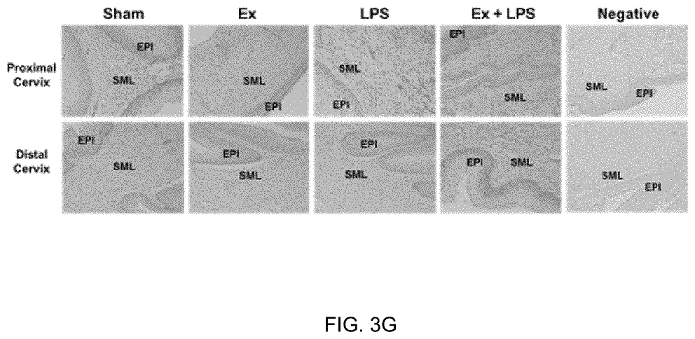

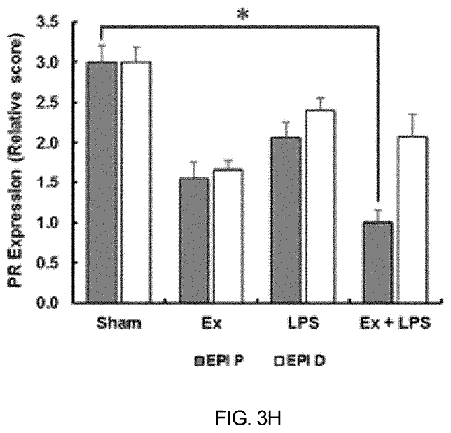

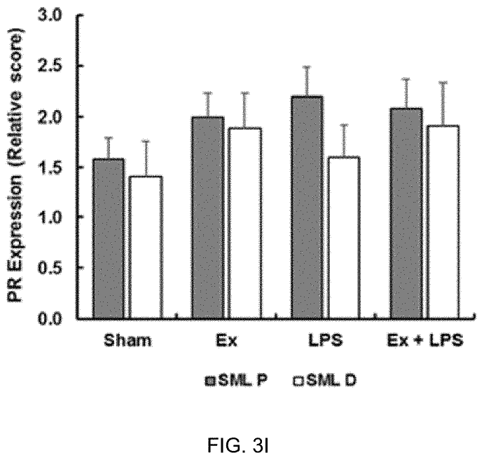

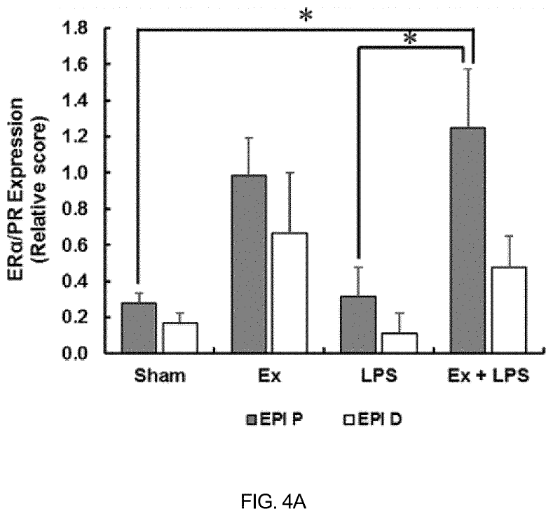

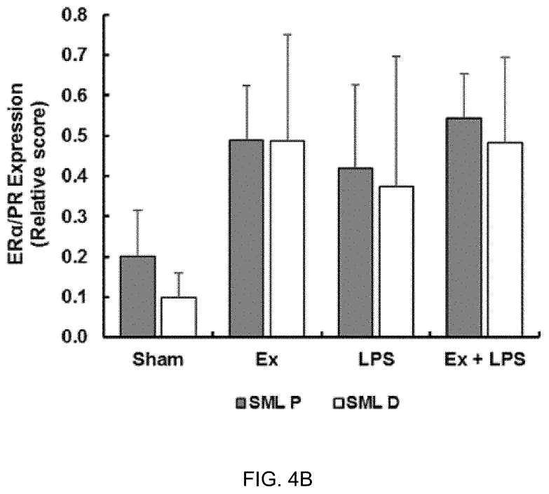

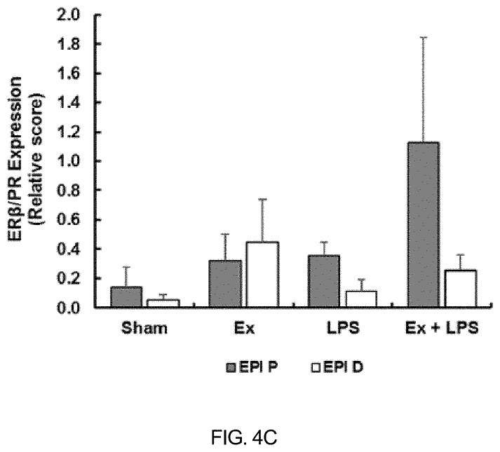

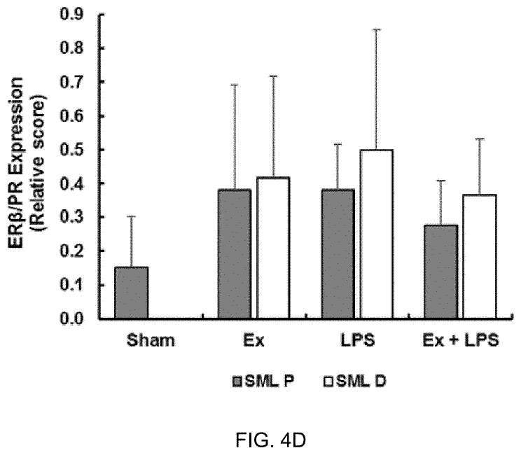

The ERα expression in the SML of the proximal cervix was significantly higher in the Ex+LPS group than in the sham group. However, there was no difference in EPI (FIGS. 3A to 3C). There was no difference in ERβ expression among groups (FIGS. 3D to 3F). The PR expression in the EPI of the proximal cervix was significantly lower in the Ex+LPS group than in the sham group. However, there was no difference in EPI of the proximal cervix (FIGS. 3G to 3I). The ratio of ER to PR (ERα/PR) in the EPI of the proximal cervix was significantly higher in the Ex+LPS group than in the sham and LPS groups (FIG. 4).

It can be seen from the above results that in preterm birth, the expression of ERα was increased in the smooth muscle layer of the proximal cervix and the expression of PR was decreased in the epithelium of the distal cervix.

4. Maternal Serum Levels of Estrogen and Progesterone

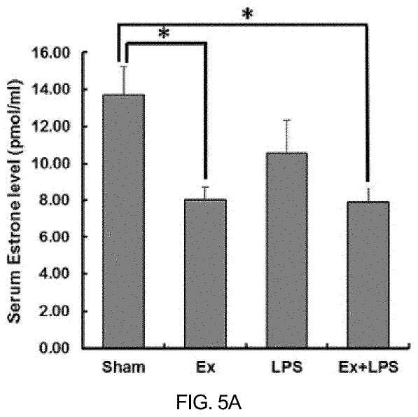

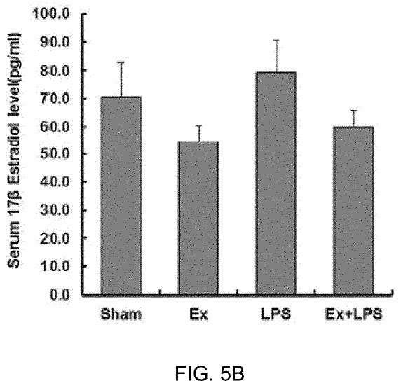

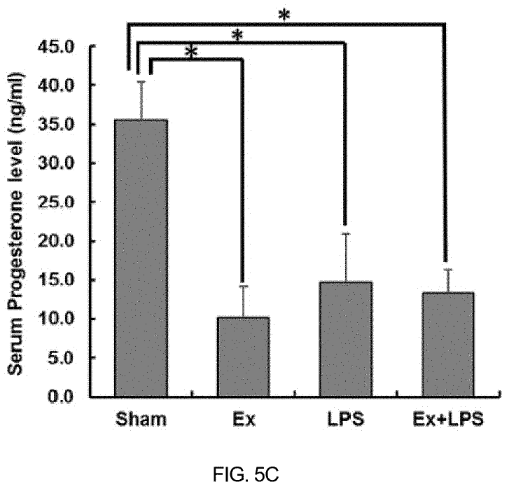

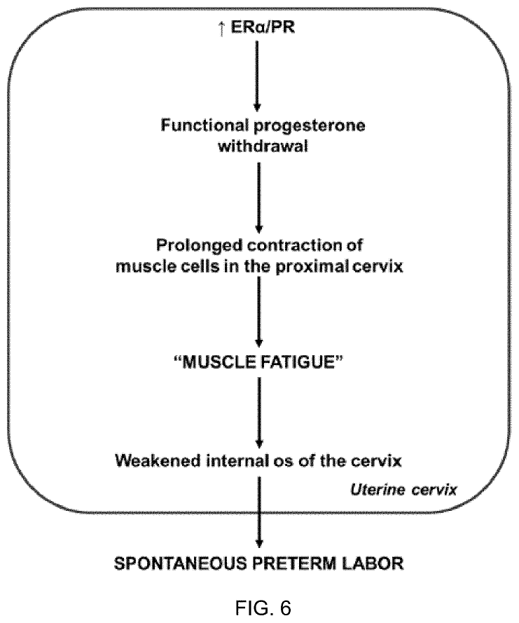

The serum estrone (E1) level was lower in the Ex group and Ex+LPS group than in the sham group (FIG. 5A). There was no difference in serum 178 estradiol level among groups (FIG. 5B). The serum progesterone (P4) level was significantly lower in the Ex, LPS, and Ex+LPS groups than the sham group (FIG. 5C).

It can be seen from the results that the maternal serum estrone (E1) and progesterone (P4) levels were reduced in preterm birth.

What is claimed is:

1. A method for providing information necessary for diagnosis of preterm birth, the method comprising:
measuring the expression level of an estrogen receptor alpha (ERα) or progesterone receptor (PR) gene in a biological sample isolated from the cervix of a subject; and
determining that the subject has a high risk of preterm birth when the expression level of the PR gene in the subject is lower than that in a normal control, wherein the expression level of the PR gene is measured in a biological sample isolated from the epithelium of the cervix of the subject.

2. A method for preventing or treating preterm birth, the method comprising:
(a) measuring the expression level of an estrogen receptor alpha (ERα) or progesterone receptor (PR) gene in a biological sample isolated from the cervix of a subject;
(b) determining that the subject has a high risk of preterm birth when the expression level of the PR gene in the subject is lower than that in a normal control, wherein the expression level of the PR gene is measured in a biological sample from the epithelium of the cervix of the subject; and
(c) performing a prevention or treatment for preterm birth on the subject determined to have a high risk of preterm birth in step (b).

3. The method of claim 2, wherein the prevention or treatment for preterm birth is performed by administration of a prevention or treatment agent for preterm birth or by a surgical intervention.

4. The method of claim 3, wherein the prevention or treatment agent for preterm birth is a tocolytic agent, an antibiotic agent, a steroid preparation, or a combination thereof.

5. The method of claim 4, wherein the tocolytic agent is a beta-sympathomimetic agent, a calcium channel blocker, an oxytocin antagonist, a non-steroidal anti-inflammatory drug, magnesium sulfate, a progesterone receptor agonist, an estrogen receptor alpha inhibitor, or a combination thereof.

6. The method of claim 3, wherein the surgical intervention is cervical cerclage.

7. The method of claim 2, wherein the measuring of the expression level of the gene is a measurement of the mRNA or protein expression level of the gene.

8. The method of claim 7, wherein the measurement of the mRNA expression level is carried out by amplifying mRNA of the gene via RT-PCR.

9. The method of claim 7, wherein the measurement of the protein expression level comprises carrying out an immunoassay of a protein.

10. The method of claim 2, further comprising determining that the subject has a high risk of preterm birth when the measured expression level of the ERα gene in the subject is higher than that in the normal control.

11. The method of claim 2, further comprising measuring the level of estrone (E1) or progesterone (P4) in a biological sample isolated from the blood of the subject.

12. The method of claim 11, further comprising comparing the level of E1 or P4 measured in the step with the level of E1 or P4 measured in a biological sample isolated from the blood of the normal control.

13. The method of claim 12, further comprising determining that the subject has a higher risk of preterm birth when the levels of estrone and progesterone in the subject are lower than those in the normal control.
